(19) United States
(12) Patent Application Publication
(10) Pub. No.: US 2011/0029025 A1
(43) Pub. Date: Feb. 3, 2011

(54) LOCKING PIN PLATE ASSEMBLY ADAPTED FOR FRACTURE FIXATION

(76) Inventor: Robert J. Medoff, Kailua, HI (US)

Correspondence Address: LADAS & PARRY LLP, 26 WEST 61ST STREET, NEW YORK, NY, 10023, US

(21) Appl. No.: 12/533,675

(22) Filed: Jul. 31, 2009

(51) Int. Cl. A61B 17/86; A61B 17/80

(52) U.S. Cl. 606/329; 606/297

(57) ABSTRACT

A lockable pin plate assembly which has a pin plate adapted to be secured to stable bone and provided with pin holes for pins for pinning an unstable bone fragment. One or more pin holes are engageable by pins of an integral U-pin. The pins have stop surfaces thereon which are resiliently displaced when the pins are seated to automatically and self-lock the pin and the pin plate to oppose withdrawal and backing out of the pins from the pin plate.

### FIELD OF THE INVENTION

The invention relates to a lockable pin plate assembly for fracture fixation which includes a pin plate adapted to be secured to stable bone and a pin member for engaging an unstable bone fragment of the fracture and securing the fragment to the plate.

In particular, the invention relates to the pin member which serves to pin the unstable bone fragment and concurrently secures the pin member in locked engagement with the pin plate.

The invention further relates to a method for pinning the unstable bone fragment by a pin member while the pin member becomes lockably secured to the pin plate. In the case of fractures of the end of a bone, a small fragment can be produced which can be secured by the invention.

### BACKGROUND

One common method of fixing fractured bones is to use a plate and screws to secure the bone fragments. In this method, screws are inserted through holes in the plate in order to secure the fragments to the plate. Although this technique is effective when the fragments are relatively large in relation to the diameter of the screw, when the fragment size is small in relation to the diameter of the screw, the size of the screw hole required in the bone fragment can weaken the fragment and cause it to fragment further, resulting in failure of fixation. Additionally, standard bone screws require thread purchase in the bone in order to compress the fragment against the undersurface of the plate in order to provide stability. If the fragments are small or consist of poor quality bone, thread purchase can be inadequate, resulting in failure of fixation.

In my previous U.S. patents (U.S. Pat. Nos. 5,931,839 and 7,044,951 plates have been disclosed that utilize small pins that are placed through the plate and into the bone fragments. This alternative type of fixation reduces the risk of causing additional fragmentation of a small fragment by reducing the hole size needed for fixation. In addition, since the pin does not depend on thread purchase in the bone fragment, this technique avoids the failure caused by poor purchase of screw threads.

Although the pin plates provide these advantages, the fixation by the pin is biomechanically different from that provided by a screw in a plate. In U.S. Pat. No. 5,931,839, the pin is not secured to the plate, but only restricts translational movement of the pin in relation to the surface of the plate. In this type of implant, axial movement of the pin in the hole as well as angulation of the pin within the hole are not constrained. If the pin is not rigidly secured into an opposite stable bone fragment, this may result in failure of fixation.

In U.S. Pat. No. 7,044,951 the pin is stabilized to the plate by modifications in the plate. Some of the modifications of the plate (FIGS. 3, 3A, 5, 6, 6A, 7, 8, 9, 10, 11, 12, 13, 14, 24, 25, 26) are constructed to provide a channel or aperture that prevents the pin from backing out of the bone. These embodiments limit axial movement of the pin but do not constrain angular movement of the pin. In other modifications (FIGS. 15, 16A, 16, 17, 18, 19, 20, 32, 33, 34, 35) the pin is captured by tabs on either side. These types of designs also limit axial movement of the pin out of the pin hole but do not limit angular movement of the pin in relation to the plate. In addition, they are cumbersome to manufacture and complicate the surgery by requiring the surgeon to bend and/or thread the end of the pin through the tabs in the plate. In still other modifications (21, 22, 23, 27, 28, 29, 31, 32, 33, 34, 25) the pin is constrained by frictional purchase of the plate against the side wall of the pin. Like the other embodiments, these modifications limit axial movement of the pin in relation to the plate, but provide less constraint to angular movement. In addition, most of these require the surgeon to crimp a portion of the plate with a bending instrument, which adds to the complexity of the procedure and may be difficult to do because of difficult access to the plate from a limited exposure. In addition, many of these embodiments require the pin to be bent or cut after the pin is in place which adds further to the complexity of the surgery. Finally, since many of these designs leave the end of the pin extending out of the plate, the cut end can cause irritation of the soft tissues and even tendon rupture.

### SUMMARY OF THE INVENTION

An object of the invention is to provide a pin plate assembly in which pins can be utilized for fixation of the bone fragment and a locking mechanism is provided between the pins and the plate to cause the pins to become locked in the pin plate when installed in the fragment.

In accordance with the invention, a locking pin plate assembly is provided which includes a pin plate adapted to be secured to stable bone and having one or more pin holes in which a pin member can be inserted to an operative position for fixation of a bone fragment and wherein a locking mechanism is provided between the pin member and the pin plate to automatically self-lock the pin member in the plate when the pin member is inserted to its operative position.

In accordance with the invention, the locking mechanism has an unlocked state in which the pin passes through the pin hole and a locked state when the pin reaches the operative position.

The pin member can be in the shape of a U with adjacent legs which form pins connected by a cross member. The legs of the U pin are inserted through the pin holes and pin the unstable fragment and serve as an integral unit which, when seated on the pin plate, lockably secure the U pin to the pin plate to oppose withdrawal of the U pin from the plate.

The invention is further concerned with a particular construction of the pin plate.

The invention is also concerned with the method of automatic self-locking of the pin in the plate upon fixation of the unstable bone fragment.

It is a feature of the invention to provide locking of the pin in the pin plate without any additional instrumentation or complicated surgical techniques.

It is a further feature of the invention to provide a locking pin plate that restricts translational, axial, and angular movement of the pin in relation to the plate.

It is a further object of the invention to provide a pin that can be simply inserted and requires no cutting of the pin and leaves no end of the pin protruding in the soft tissue.

In further accordance with the invention, the pin and the hole are formed so that when the pin is in its operative position engaging the bone fragment, a clearance is formed between the pin in the hole and a force is produced on the pin to displace the pin in the hole to a lock position in which the pin is not removable from the hole

In further accordance with the invention, the cross member of the U pin provides flexibility to develop the force to displace the legs of the pin.

In further accordance with the invention. the legs of the U-pin are provided, on external surfaces thereof, with grooves or ridges defining shoulders at the ends of the grooves which, when the U pin reaches its operative position seated on the pin plate, the legs flex and lock the shoulders at the end of the grooves or ridges against a surface of the pin plate to lock the U pin in the pin plate and oppose its removal.

In further accordance with the invention, the U pin may also be formed to have different diameters over its length. For instance, a larger diameter portion may be preferred for an intraosseous portion of the pin in order to provide greater strength to cantilever bending, while a smaller diameter portion may be preferred for an extraosseous portion of the pin in order to avoid soft tissue irritation.

A typical sequence of insertion is to first apply and secure the plate to the stable bone fragment. A removable pin is then drilled through the pin holes in the plate and into the unstable bone fragment to form pilot holes in the fragment. An appropriate size and contoured pin is then selected and impacted through the pin and pilot holes and into the unstable fragment. Impaction of the pin results in automatic, self-locking of the pin to the plate.

One typical application of this construction is for fractures of the medial malleolus of the ankle and is described herein. However, this is purely exemplary and other bone fractures are equally applicable, such as, the distal radius or distal humerus, spine and other bones. In addition, although the examples used herein describe a plate with the locking pins at one end, this type of locking pin plate design is equally applicable for placement in any position over the length of the plate. For example, locking pins could be used in the central portion of a plate for fixation of shaft fractures or over the entire length of the plate for stabilization of spinal elements.

### BRIEF DESCRIPTION OF THE FIGURES OF THE DRAWING

FIG. 1 is a diagrammatic, side elevation view, partly broken away, showing the installation of the pin plate assembly on a fractured bone of the ankle;

FIG. 2 is a perspective view of the installation shown in FIG. 1;

FIG. 3 is a side elevational view of the pin plate of the pin plate assembly;

FIG. 4 is a top perspective view from the right, of the pin plate in FIG. 3;

FIG. 5 is a front elevation view of a U pin of the pin plate assembly;

FIG. 6 is a side elevation view of the U pin;

FIG. 7 shows, in end view, the installation of the U pin in the pin plate before the U pin is fully seated on the pin plate;

FIG. 8 is similar to FIG. 7 after the U pin is fully seated on the pin plate; and

FIG. 9 is an end view of the pin plate from the right side of FIG. 4.

FIGS. 10-16 diagrammatically illustrate variations of the pin and pin plate assembly.

### DETAILED DESCRIPTION OF THE INVENTION

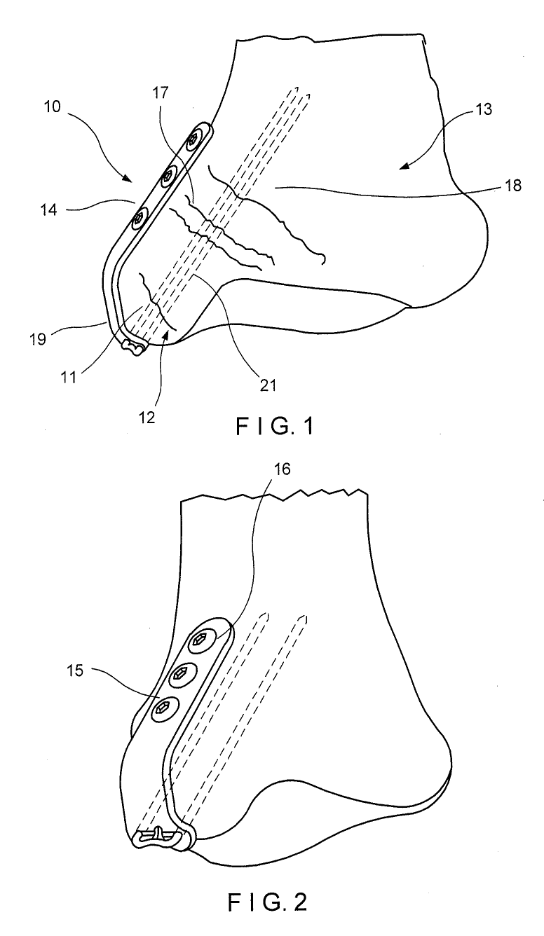

Referring to FIGS. 1 and 2 therein is shown a pin plate assembly 10 adapted for fixation of a small fragment 11 of a bone fracture 12 of the medial malleolus of the distal tibia bone 13 which is adjacent to the ankle.

Although the pin plate assembly 10 is described for fixation of the fracture 12 of the ankle bone 13, this is for exemplary purposes only and the pin plate assembly is applicable to fractures of all bones.

By way of example of other fracture sites are the lateral/medial condyle or epicondyle of the elbow, proximal shoulder, distal fibula, olecranon, proximal/distal radius, distal ulna, and even metacarpal/metatarsal bones or phalangeal bones of the hand or foot or stabilization of spinal elements.

The pin plate assembly comprises a pin plate 14 having a first portion 15 with holes 16 adapted for receiving at least one fastener 17 in the form of bone screws for securing the first portion to stable bone 18. Although the expression “bone screws” is used for simplicity in the description of securing the plate to bone, the fastener 17 is not limited to screws. In other embodiments pins, wires, blades, staples, brackets, or indirect coaction with another device securely attached to the stable bone fragment through holes in the plate are used.

The pin plate 14 is shaped to fit on the bone and includes a second portion having pin holes 20 at the distal end adapted for receiving at least one pin 21 for engaging the unstable bone fragment 11. In the illustrated embodiment, the pin 21 pins the unstable fragment to stable bone 18. However, as will be seen later, the pin may only be pinned to the unstable fragment.

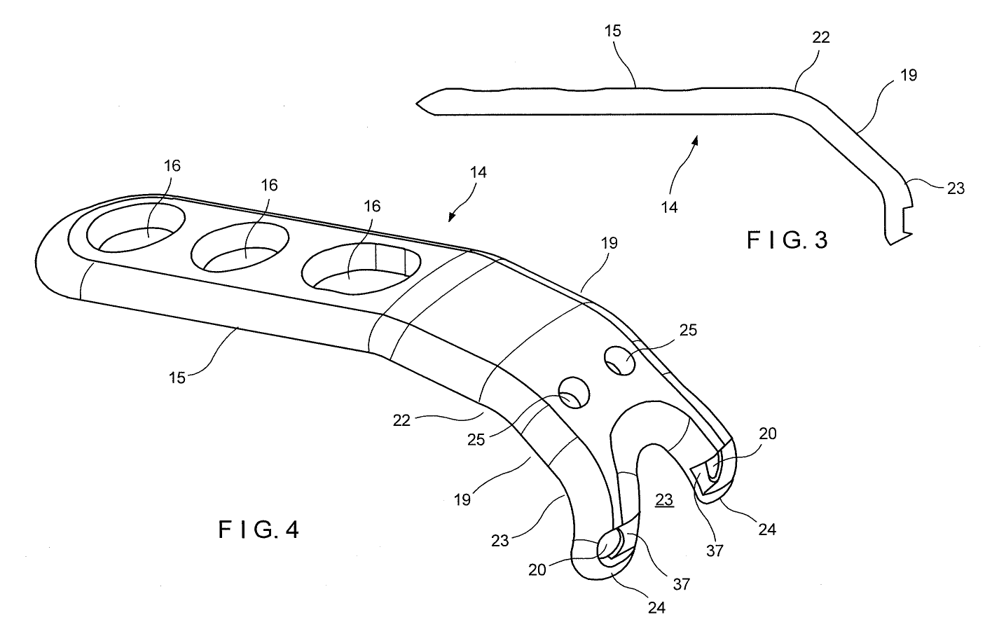

As best shown in FIGS. 3, 4 and 9, in one embodiment the pin plate has a first bend region 22 of about 45□ and a second bend region 23 so that the distal end of the pin plate is predominantly perpendicular to the longitudinal axis of the pin plate.

As best seen in FIGS. 4 and 9, the distal end of the pin plate 14 has a U shaped opening 23 which forms legs 24 on opposite sides of the opening 23. The legs 24 are slightly flared away from one another to provide an increased spacing of the pin holes 20. A pair of guide holes 25 is provided in the plate more proximal than the pin holes 20 and can serve for installation of conventional pins or bone screws for temporary fixation of the bone, or as an attachment site for a drill guide.

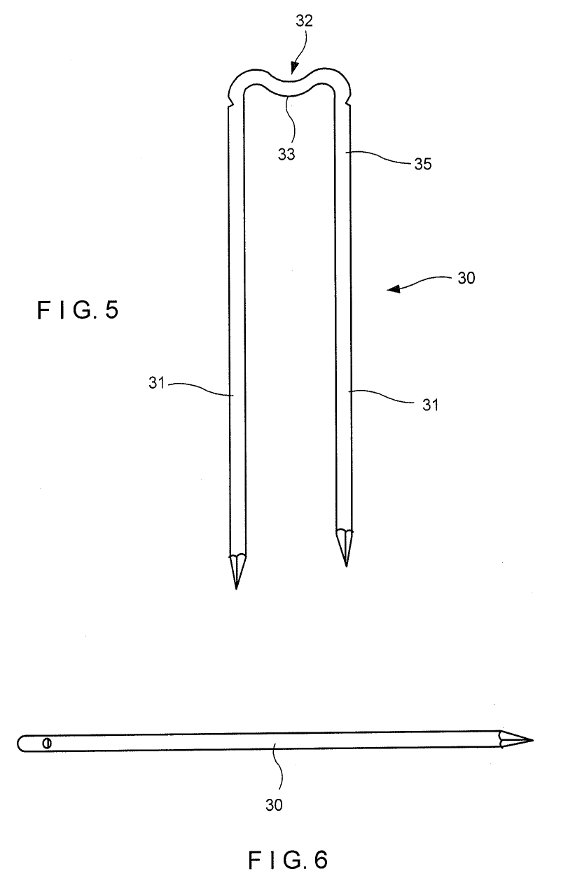

In order to pin the unstable bone fragment, a pin member in the form of a U as shown in FIG. 5 and hereinafter referred to as a U-pin 30 is utilized. The U pin 30 has a pair of generally parallel legs 31 forming pins which are connected by a cross piece 32 to provide the base for U pin 30. In the illustrated embodiment the cross-piece 32 is formed by a bend which provides additional flexibility of the legs at the ends of the bend where they are joined to the legs.

The U pin 30 is formed as a bent wire and has a diameter based on the bone being pinned. Alternatively, but not shown, the U pin may have differing diameters between a portion of the leg members 31 and a portion of the cross-piece 32. The diameter of pinning members is well known to those skilled in the art. The pin plate 14 has a size related to the bone being repaired and in general can vary from a thickness of 0.020 to 0.250 inches. In general, the pin plate is relatively stiff and resists bending. However, under certain circumstances, the pin plate can be made flexible so that the pin plate is pushed against the bone as it is screwed into the bone.

The pin and the pin plate are made of conventional material such as stainless steel, titanium or titanium alloys, PEEK, or other suitable polymers and bioabsorbable material.

The ends or tips of the pins 31 are pointed to facilitate penetration of the tips into bone. In another embodiment, the tips are bullet shaped.

The cross piece 32 has an inward dimple 33 which serves a purpose to be explained later. The dimple 33 also provides flexibility at the base of the U pin also for a purpose to be explained later.

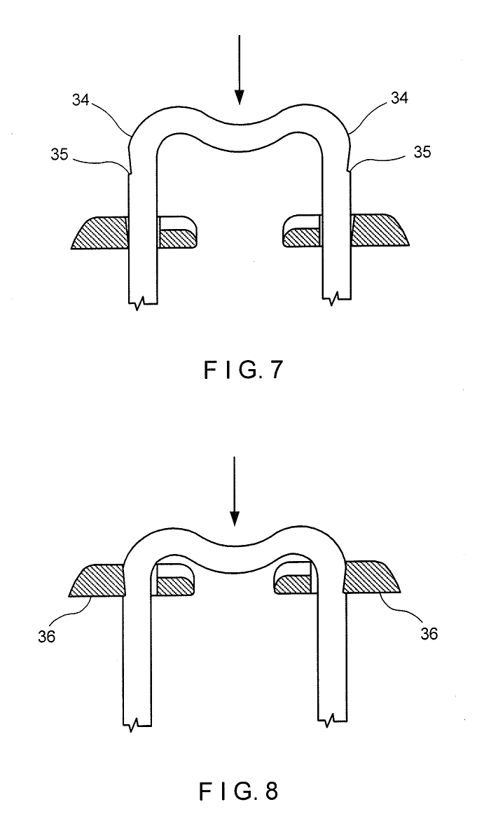

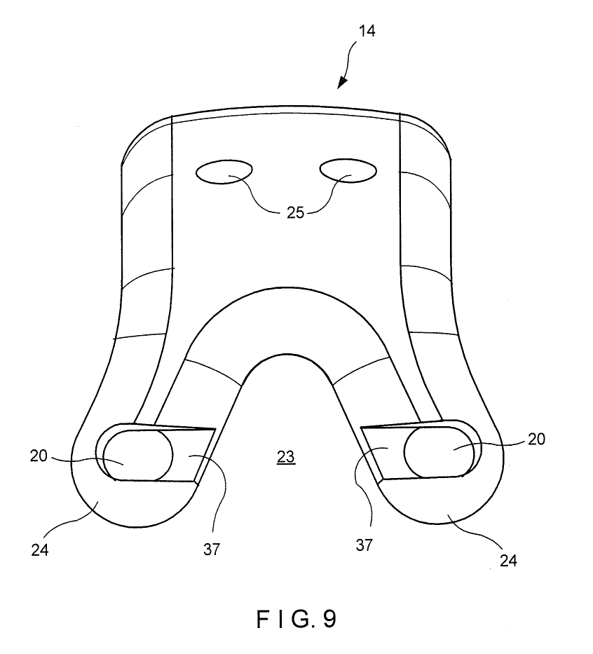

The U pin 30 is provided with grooves or notches 34 on the outer surfaces of the legs 31 extending downwardly from the cross piece 32. The grooves form stop surfaces serving as shoulders 35 or shelves at the ends of the grooves 34. The grooves 34 and shoulders 35 are best seen in FIGS. 7 and 8. The grooves 34 and shoulders 35 form part of the locking mechanism for automatically self-locking the pin in the pin holes in the plate. Although shown in this drawing on the outer surface of the pin, they could be formed on any external surface of the pin. Instead of forming grooves or notches on the outer surfaces of the pins, the pin surfaces can be crimped to form indentations along one cross-sectional axis and produce shoulders 35 along the perpendicular cross-sectional axis. The width of the cross piece or bend of the U pin is equal to or slightly greater than the spacing between the pin holes 20 in the legs 24 when the grooves are on the outer surfaces of the legs 31 and when the grooves are on the inside surface of the legs, the spacing is equal to or slightly less than pin spacing.

In order to achieve fixation of the fracture, first the fracture is reduced and then the bone screws 17 are utilized to secure the pin plate to stable bone. Two pilot holes are then drilled in the unstable fragment through the pin holes 20. In the case of the medial malleolus, the pilot holes only need to penetrate the cortex of the unstable fragment whereafter the U pin is then impacted into the bone fragment by simple hammering it in.

Referring to FIGS. 7 and 8, it is seen in FIG. 7 that before the U pin is fully seated in the pin plate, the legs 31 of the U pin tightly fit in the pin holes and resiliently bear against the outer walls of the pin holes due to the resilience afforded by the cross piece as well as angular bends in the pin and the spacing of the legs with respect to the spacing of the pin holes. In a preferred embodiment, the spacing between the legs 31 in a rest state is slightly greater than the spacing between the holes 20 in the plate so that the legs 31 of the U pin are squeezed together under tension in order to engage the holes 20. When the grooves 34 on the outer surfaces of the legs 31 enter the pin holes and pass therein and the U pin is fully seated in the pin holes, as shown in FIG. 8, the pins automatically snap outwardly to position the shoulders 35 beneath the edge of the undersurface 36 of the pin plate. This securely locks the U pin in place on either side in the pin holes 20 in the legs 24 of the pin plate. In this way, the U pin cannot back out of the pin plate and a secure locking engagement of the U pin in the pin plate is achieved.

In a preferred embodiment, the diameter and direction of holes 20 in plate 14 are directed for optimal fixation at the site of application. The orientation of the hole may be designed to optimally locate the legs 31 of U pin 30 in the unstable bone fragment. In addition, the difference between the inner diameter of holes 20 in plate 14 and the outer diameter of legs 31 of U pin 30 is preferably large enough to allow passage of the legs 31 through the holes 20, yet with tolerances that are tight enough when the pin is fully seated to limit angular movement of the legs 31 in holes 20.

Although the grooves 34 have been shown on the outer surfaces of the pins it is equally possible to place the grooves on the inner or outer surfaces of the pins. If placed on the inner surfaces of the legs 31 of U pin 30, the width of the bend at the cross piece of the U pin would be made equal to or slightly smaller than the spacing between the pin holes 20 so the pins will snap inwardly to lock the shoulders 35 under the plate. In an alternative embodiment, the groove may be present on only one leg of U pin 30.

Although pin plate assembly 10 is illustrated with the locking U pin at one end of the pin plate and the bone screws at the other end, it is possible to have locking U pins in a central portion of the plate, at either or both ends of the plate, or throughout the length of the plate. In addition, although pin plate assembly 10 is demonstrated with bone screws at one end, it is possible to have a pin plate assembly in which no bone screws are used but rather the plate is secured only with locking pins at either end or centrally in the plate.

The dimple 33 which faces inwardly between the legs provides slight resilience for the cross piece to enhance the resilient force on the pins and promote the snap engagement of the shoulders 35 with the undersurface 36 of the pin. Alternatively, the cross piece between the legs 31 of the U-pin 30 can be bent outwardly from the plane formed by the legs 31 of U-pin 30. By varying the length of the cross piece out of the plane of legs 31 and the diameter of the cross piece the amount of resilience can be varied to provide a lateral force of the pin on the side wall of the pin hole that is appropriate for the site of application.

The pin plate 14 is provided with grooves or cutouts 37 (note: I can't find number 37 on the figures) as shown in FIGS. 4 and 9 that extend from the pin holes 20 to the U shaped opening 23. The cutouts 37 are shaped and dimensioned to allow the cross piece 32 of the U pin to seat almost flush against the end of the pin plate when fully seated thereby avoiding any protruding corners or sharp ends that might irritate soft tissue. In addition, the dimple 33 allows the cross piece to sit flush against the bone surface between the legs 24 and avoid irritation of soft tissues.

In prior pin plate assemblies, there was little angular stability of the pin in the pin hole of the plate. In contrast, the pin plate configuration of the current invention is substantially stable and as previously recited; the U pin will not back out of the pin plate. Additionally, since the engagement of the pins in the pin holes is achieved with a single U pin engaged in two distinct pin holes separated by a fixed distance, the U pin is rigidly held by the pin plate and won't angle from side to side. In addition, the relatively tight fit of the pin within the hole of the pin plate coupled with the pressure of the pin against the side wall from the resilience of the cross piece 32 as well as the lock of the shoulder against the edge of the plate serves to restrict angular movement of the pin the hole. With the present invention the U pin which is a single structure that is held relatively rigidly in the holes by the lock between the shoulder 35 against the undersurface 36 of pin plate 14. Because of this intrinsic stability, there is no need to capture a far cortex with the pin but rather one or both legs of the U pin can terminate within the metaphyseal bone. Thus, pinning of the bone fragment only requires the pin to extend into the fragment and not through the fragment and into and through stable bone. This greatly simplifies the surgical technique since the surgeon does not have to measure the pin length, withdraw the pin, cut the end of the pin to length and form a hook on the end of the pin and then re-impact the pin into the bone. Instead, according to the invention, the surgeon simply drills the pilot holes and hammers the U pin in place thereby providing automatic locking of the shoulders against the undersurface of the pin plate at the edges of the pin holes and integral locking of the pin with the plate.

In essence, it is the flexibility of the cross piece 32 and the angular bends of the U-pin 30 and the grooves 34 with shoulders 35 that snap into their respective pin holes 20 and lock the U-pin to the pin plate. Hence, the locking mechanism between the pin and the pin plate is initially in unlocked state and the pin passes through the pin hole to the operative position whereupon the pin is shifted and the locking mechanism automatically self locks the pin in the pin plate in locked state. This allows the pin to be slightly oversized or undersized so that as the pin is seated, the tendency to return to the initial position of the U pin, engages the shoulder 35 against the undersurface of the plate.

Although the U-pin has been shown as having two legs or pins 31, it is also possible to utilize only a single pin with at least one leg formed with shoulder 35 to lock the pin in the pin plate. In such case, the locking pin would have an L-shape, J-shape, or shape similar to a nail. Accordingly, even though the pin member has been referred to as a U-pin, it is to be understood that this term also refers to other shapes as described above.

In addition, although the current description shows a plate 14 with two holes 20 for engagement of the two legs 31′ of U-pin 30, it is also possible to have a plate with a single hole for one leg 31 of U-pin 30, with the other leg abutting against the side of the plate.

The current description demonstrates a locking mechanism in which the shoulder 35 of groove 34 of U-pin 30 that locks against the bottom edge of the pin hole 20 in the plate. It is also evident that this locking mechanism could be designed with an edge or ridge within the pin hole as well, without affecting the spirit of the invention. For instance, a ridge within the hole could engage the groove on the U-pin.

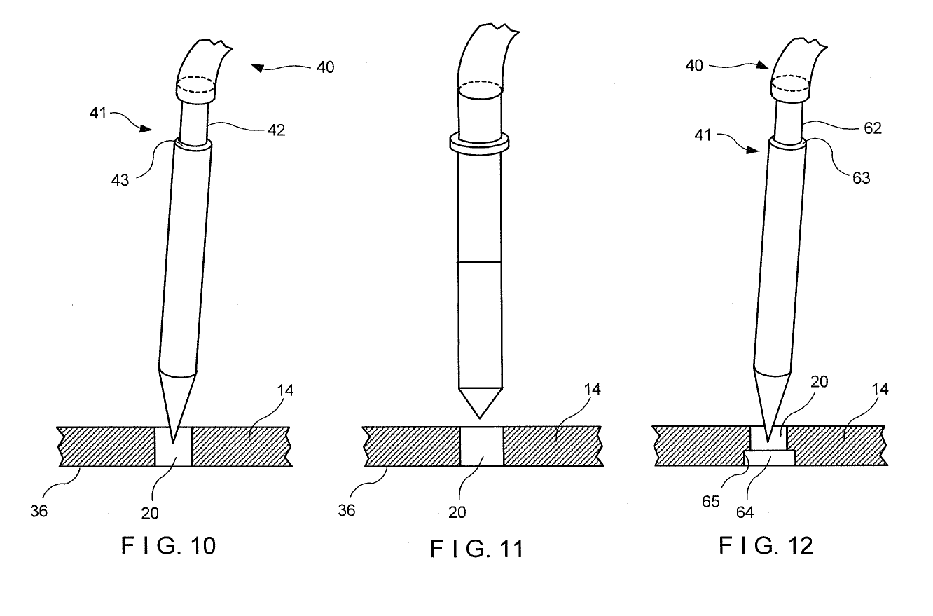

In the disclosed embodiments, the lateral displacement of the pin in the hole is provided by the flexibility of the pin itself and its elastic deformation when the pin is relaxed to shift the stop surface into engagement with the pin plate to lock the pin in the pin hole. In another arrangement, the pin hole can be made elastic as for example shown in my published application (Ser. No. 11/103,923) so that when a slightly oversized pin is inserted into the hole, the wall of the hole will yield and when the portion with the groove reaches the bottom of the hole, the wall itself will relax and urge the pin laterally against the wall and lock the stop surface under the plate. FIG. 10 illustrates a variation of the pin shown in FIGS. 7 and 8. Instead of forming a groove at one side surface of the pin, as in FIGS. 7 and 8, the leg 41 of pin 40 is formed with a reduced cylindrical portion 42 forming an annular shoulder 43. The installation and engagement of pin 40 in the pin hole 20 in pin plate 14 to automatically self-lock the pin 40 in the plate 36 serves to prevent its withdrawal and backing out of the hole similar to FIG. 8.

FIG. 11 shows another variation. Therein, the leg 51 of pin 50 is formed with a ridge 52 partially or completely around the pin. The outer diameter of ridge 52 corresponds to the inner diameter of pin hole 20 in plate 14 but is slidable therein. The diameter of the remainder of the shaft of leg 51 is less than the diameter of ridge 52. As before, the pin is driven through the pin hole into the bone fragment. Near the end of the travel of the pin, the ridge 52 enters the pin hole and passes therethrough whereupon, the pin is laterally displaced by the resilient force of the pin member to automatically engage the ridge 52 under the undersurface 36 of pin plate 14 to lock the pin and prevent its withdrawal from the hole.

FIG. 12 shows another variation of the engagement of the U-pin 40 and pin plate 14. Therein, the pin 41 is formed similarly to FIG. 10 with a reduced diameter portion 62 forming an annular shoulder 63. The pin hole 20 in pin plate 14 is formed with a countersink 64 at its lower end to form a ledge 65 at the juncture between pin hole 20 and countersink 64. As in the previous arrangements, the pin is driven through the pin hole 20 into the bone fragment. When the shoulder 63 passes the ledge 65, the pin is elastically urged outwards (to the left in FIG. 12) and the shoulder snaps beneath the ledge to automatically lock the pin in the pin hole to prevent its withdrawal and backing out from the hole. Because the shoulder 63 engages the recessed ledge 65 in hole 20, the reduced diameter portion 62 is less in length than that in FIG. 10 by an amount substantially equal to the depth of countersink 64.

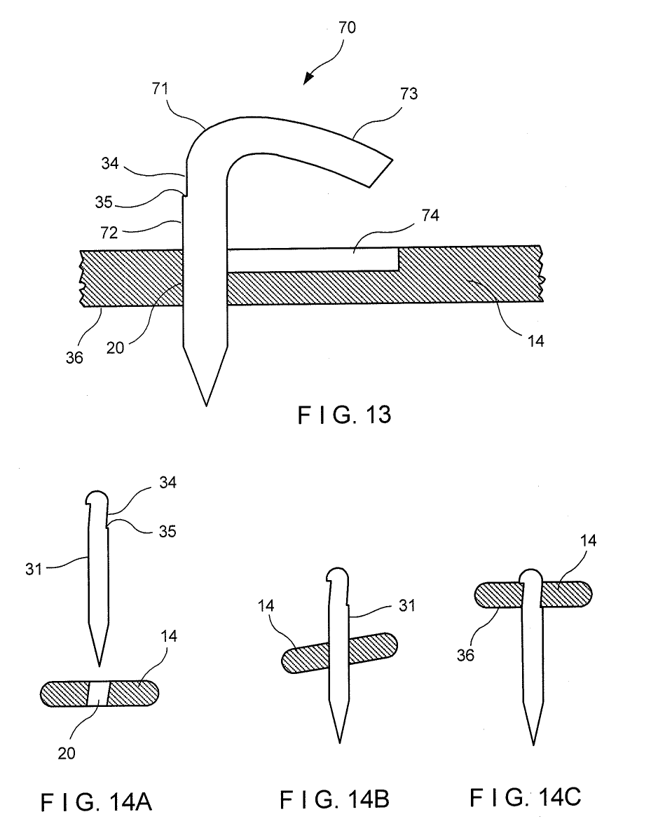

FIG. 13 shows another embodiment of a pin 70 for automatically locking in a pin plate 14. In this embodiment, the pin 70 is bent at bend 71 to form legs 72 and 73. The legs 72 and 73 form an acute angle therebetween. The plate 14 is formed with a recess 74 in its upper surface similar to the cut-out 37. The outer surface of leg 72 is formed with a groove 34 forming a shoulder 35 as in the earlier described embodiments. As before, the leg 72 of pin 70 is driven through pin hole 20 in pin plate 14 into the bone fragment to be pinned. When the groove 34 enters the pin hole, the leg 73 contacts the pin plate at the bottom of recess 74. As the pin is further advanced in the pin hole, the leg 73 flattens out to increase the angle between the legs 72 and 73 and apply a resilient force on leg 72 to press leg 72, against the outer wall of hole 20. When the shoulder 35 passes the lower surface 36 of plate 14, the shoulder 35 snaps under surface 36 to automatically lock the pin in the pin plate.

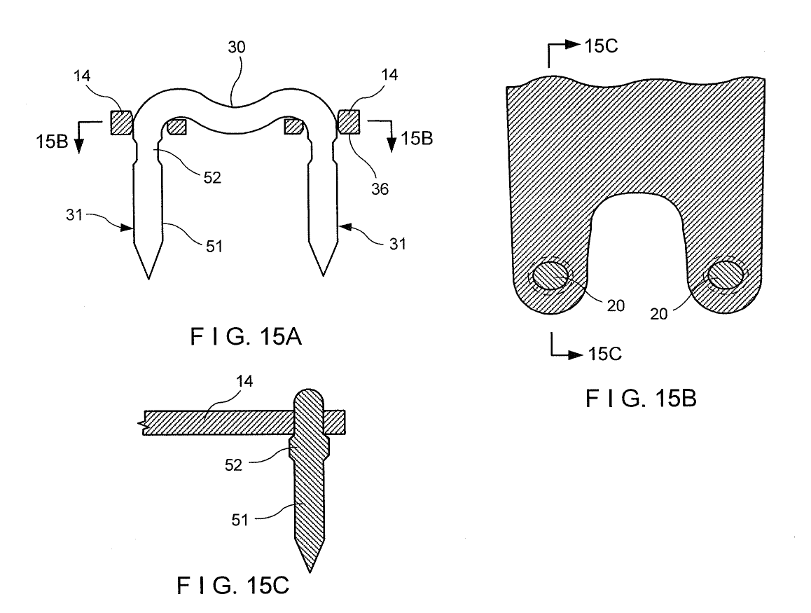

FIGS. 14A-C illustrates an embodiment in which the plate exerts resilient force to shift the pin laterally to its locked position. Referring to FIG. 14A, therein is seen pin 31 with groove 34 therein in readiness to be inserted into hole 20 in pin plate 14. In contrast with the previous embodiments, the hole 20 is inclined in the pin plate. FIG. 14B shows the pin 31 partially inserted into hole 20. Due to the inclination of the hole, when the pin is driven straight in, perpendicular to the plate, the plate is elastically deformed as shown in FIG. 14B. When the groove 31 on the pin passes through the hole and shoulder 35 exits from the bottom of the hole, the plate 14 returns to its initial state and urges the pin laterally to engage the shoulder 35 under the undersurface 36 of the plate to lock the pin against withdrawal and backing out from the hole

In the previous embodiments, the pins are cylindrical, the holes are circular, and the stop surface is formed by a shoulder at the bottom of a groove in the external surface of the pin or by a ridge that is seated against the undersurface of the plate. However, the pins can have different cross-sectional configurations, the holes can be non-circular and the stop surfaces can be formed as part of the cross-section of the pin as shown hereafter.

FIGS. 15A-C show an embodiment in which U-pin 30 is installed in pin plate 14 and where the legs 31 of U-pin 30 are crimped rather than formed with grooves. In particular, each leg 31 has a first portion 51 and a second portion 52, portion 52 being crimped to define an elongated cross-section of substantially elliptical shape, while the first portion 51 has a circular shape. When the legs 31 of the pin 30 are driven through the pin holes 20 and into the bone fragment, the circular portion 51 passes through the hole. The length of the major or long axis of the elliptical cross-section of portion 52 is equal to the diameter of the hole 20. When the top of elliptical portion 52 passes through the bottom of the hole, the elastic force of the U-pin forces the legs 31 outwardly to automatically cause the tips of the elliptical portion to shift laterally behind the undersurface of the pin plate 14 and form a stop surface to prevent withdrawal or backing out of the pin from the hole as shown in FIG. 15C. The shift of the pin in the hole can be of the order of a few thousandths of an inch to provide locking of the pin legs in the holes.

In a variation, the pin hole 20 can be elliptical in cross-section with the short axis of elliptical hole 20 corresponding to the long axis of elliptical portion 52. After the elongated axis of portion 52 passes beyond the short axis of elliptical hole 20, the leg 31 can laterally displace to the tip of the long axis of pin hole 20 thereby locking the elongated portion 52 under the surface of the plate. In another modification, the elliptical portion 52 can be oversized in a circular pin hole. The pin hole elastically expands slightly when the elliptical portion 52 passes through the hole, and when the elliptical portion exits from the bottom of the hole, the hole relaxes to its initial state and the circular portion 51 is automatically displaced laterally to bear against the side of the hole and the elliptical portion 52 is locked behind the bottom of the pin plate.

Preferably, in the above variations, the legs 31 of the U-pin are made divergent to produce tension in the pin and enhance the elastic action to displace the legs against the wall of the hole when the pin reaches its operative position.

In the embodiment of FIGS. 8 and 10-14 the operative position is reached when the grooved portion of the pin leg is in the pin hole, whereas in FIG. 15 and its variants, the operative position is reached when the crimped portion has passed through the hole in the pin plate.

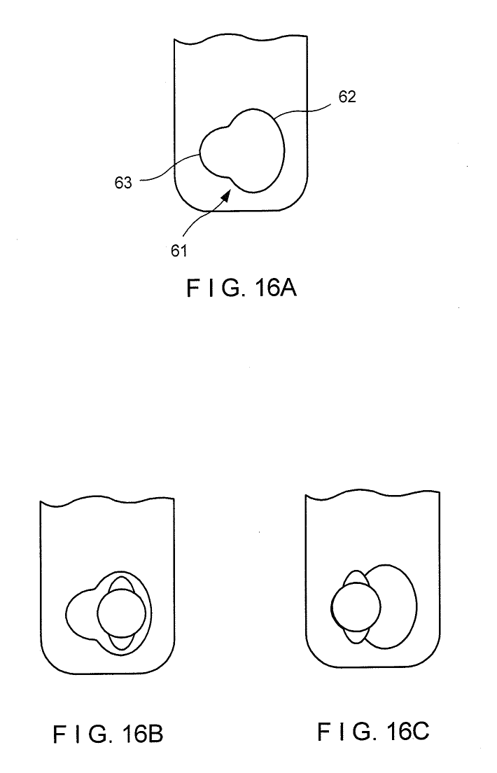

FIGS. 16A-C show another arrangement in which the pin hole 61 in the pin plate 14 has the shape illustrated in FIG. 16A. In particular, the pin hole 61 has a part elliptical cross-section 62 with a part-circular extension 63 at one side thereof. The pin has a circular cross-section in portion 51 and an elliptical crimped portion 52 as shown in FIG. 15A.

The pin is driven through the hole and the elliptical portion 52 aligns in the elliptical portion 62 of the hole as shown in FIG. 16B. When the crimped portion 52 passes the bottom of the hole, the pin is automatically displaced laterally so that the circular portion of the pin fits into the circular part 63 of the hole whereupon the tips of the crimped portion 52 are positioned behind the bottom surface of the pin plate to lock the pin to prevent its withdrawal and backing out of the pin plate.

In order to expedite insertion of the U-pin into the pin holes, the cross piece 32 can be located outside the plane of the pins in all of the disclosed embodiments . . . .

Although the invention has been described in relation to specific embodiments thereof it would become apparent to those skilled in the art that numerous modifications and variations can be made within the scope and spirit of the invention.

1. A pin plate assembly comprising:
a pin plate adapted for being secured to a bone, said pin plate having at least one pin hole,
a pin member including a pin,
said pin being insertable in said at least one hole to an operative position in which the pin engages the bone, and
a locking mechanism between the pin and the pin plate to automatically self-lock the pin in the pin plate when the pin is inserted to said operative position and oppose withdrawal of the pin member from the pin plate.

2. The pin plate assembly as claimed in claim 1, wherein said pin hole and said pin are shaped and dimensioned to automatically lock said pin in said pin hole when the pin is in said operative position.

3. The pin plate assembly as claimed in claim 2, wherein said locking mechanism includes means for applying a resilient force on said pin urging the pin against a wall of the hole.

4. The pin plate assembly as claimed in claim 3, wherein said pin is laterally displaceable in said hole and is resiliently urged to self-lock the pin in the hole when the pin is in said operative position.

5. The pin plate assembly as claimed in claim 4, wherein said pin has first and second portions along the length of the pin with different cross-sections.

6. The pin plate assembly as claimed in claim 5, wherein said different cross-sections define a stop surface on said pin which engages a surface of the pin plate to oppose withdrawal of the pin from the pin plate when the pin is in said operative position.

7. The pin plate assembly as claimed in claim 6, wherein said first portion of the pin is shaped and dimensioned to fit in said pin hole, said second portion of the pin being shaped and dimensioned relative to said hole to enable the lateral displacement of the pin in the hole, said second portion being disposed in said hole with the pin in said operative position, said means for applying resilient force then acting to laterally displace the pin to engage said stop surface on the pin and the surface of the pin plate to lock the pin against withdrawal from the pin plate.

8. The pin plate assembly as claimed in claim 7, wherein said first portion and said hole have predominantly round cross-sections and said second portion has a predominantly round cross-section with a groove therein.

9. The pin plate assembly as claimed in claim 7, wherein said first portion and said hole have round cross-sections and said second portion has an elongated cross-section.

10. The pin plate assembly as claimed in claim 7, wherein said first portion has an elongate cross-section and said second portion has a predominantly round cross-section, said hole having a cross-section in part corresponding to said elongated cross-section and in part to said round cross-section.

11. The pin plate assembly as claimed in claim 7, wherein said first portion and said hole have an elongate cross-section and said second portion has a predominantly round cross-section.

12. The pin plate assembly as claimed in claim 1, wherein said pin plate assembly is adapted for bone fixation, said pin plate being adapted for fixation to stable bone and said pin is adapted to engage a bone fragment for fixation thereof.

13. A pin plate assembly as claimed in claim 3, wherein said means for applying force includes a portion of said pin that exerts a force on the pin.

14. A pin plate assembly as claimed in claim 13, wherein said portion of the pin extends outwardly of the pin hole.

15. A pin plate assembly as claimed in claim 14, wherein said portion of the pin has resilience.

16. A pin plate assembly as claimed in claim 15, wherein said portion of the pin includes a cross piece that connects said pin with a second pin of the pin member.

17. A pin plate assembly as claimed in claim 16, wherein said second pin is insertable into a respective said pin hole in the pin plate and includes a respective said locking mechanism to oppose withdrawal of the second pin from its respective pin hole.

18. A pin plate assembly as claimed in claim 17, wherein said first and second pins have a spacing which is different from a spacing of the pin holes in the plate, said first and second pins being capable of resilient displacement for insertion into the pin holes.

19. A pin plate assembly as claimed in claim 18, wherein the cross-piece has a diameter different from the diameter of the pins.

20. A pin plate assembly as claimed in claim 1, wherein the pin is slidable in the pin hole and is substantially restrained by the plate in translation in the plane of the plate and in angulation in the pin hole.

21. A pin plate assembly as claimed in claim 1, wherein said pin member includes two said pins and a cross piece joining said pins, to form the shape of a U-pin.

22. A pin plate assembly as claimed in claim 21, wherein said pin plate has an end with a U-shaped opening therein defining spaced legs on opposite sides of the U-shaped opening, said pin holes being in said legs.

23. A pin plate assembly as claimed in claim 22, wherein said cross piece of said U-pin has a dimple therein to fit in said opening between said legs of the pin plate when the U-pin is seated on the pin plate in said operative position.

24. A pin plate assembly as claimed in claim 1, wherein said locking mechanism has an unlocked state in which said pin passes through said pin hole and a locked state when said pin reaches said operative position.

25. A pin plate assembly as claimed in claim 24, wherein said pin has a stop surface which engages said plate in said locked state.

26. A pin plate assembly as claimed in claim 24, wherein said lock mechanism shifts said pin in said pin hole to said locked state.

27. A pin plate assembly as claimed in claim 1, wherein said pin hole in sand plate is at an angle relative to a to a line perpendicular to the plate.

28. A U-pin for engagement in a pin plate to pin an unstable bone fragment, said U-pin comprising:
two legs joined by a cross-piece, said legs being engageable in respective spaced pin holes in a pin plate with the cross-piece spanning the distance between the pin holes, with at least one of said legs having
first and second portions of different cross-sectional shape defining a stop surface therebetween, said pin providing lateral resilience for said legs, said first portion of the leg being shaped and dimensioned to pass through the respective pin hole whereas the second portion enables lateral displacement of the pin in the hole due to said lateral resilience and shift of said stop surface to a locking position opposing withdrawal of the pin from the hole.

29. The U-pin as claimed in claim 28, wherein said cross piece is curved to allow said legs of said pin to be compressed or expanded for insertion into one or more pin holes.

30. The U-pin as claimed in claim 28, wherein said legs are predominantly parallel or slightly converge or diverge.

31. The U-pin as claimed in claim 28, wherein said legs lie in a plane and said cross-piece lies in a plane offset therefrom.

32. A method for fixation of a bone fracture comprising the steps of:
securing a pin plate to bone, said pin plate having at least one pin hole,
impacting a pin through said at least one pin hole into a fragment of the fractured bone for fixation thereof, and
forming said pin and said pin hole so that when the pin is in the plate and
impacted into the bone fragment to an operative position of the pin in the plate, the pin automatically self-locks in the pin plate to oppose backing out of the pin from the pin plate.

33. The method as claimed in claim 32, comprising forming said pin with a stop surface that engages the pin plate when in said operative position therein to self-lock the pin in the pin plate.

34. The method as claimed in claim 33, comprising applying resilient force on the pin to urge the pin to engage its stop surface with the pin plate when in said operative position in the pin plate.

35. The method as claimed in claim 34, comprising providing clearance of said pin in said pin hole when the pin is in said operative position in the pin plate, and displacing said pin by said resilient force to engage said stop surface with the pin plate.

36. The method as claimed in claim 34, comprising providing two said pins connected by a cross-piece forming the shape of a U-pin and inserting said two pins of said U-pin into respective pin holes in said pin plate.
